(19) United States
(12) Patent Application Publication
(10) Pub. No.: US 2025/0216391 A1
(43) Pub. Date: Jul. 3, 2025

(54) METHOD FOR PREDICTING PATIENT RESPONSE TO IMMUNOTHERAPY

(71) Applicant: ASTRAZENECA AB, Södertälje (SE)

(72) Inventors: Ikbel ACHOUR, Wilmington, DE (US); Thomas HERZ, Munich (DE); Lina MEINECKE, Munich (DE)

(21) Appl. No.: 18/844,266

(22) PCT Filed: Mar. 6, 2023
(86) PCT No.: PCT/EP2023/055633

§ 371 Date: Sep. 5, 2024

Related U.S. Application Data
(60) Provisional application No. 63/317,259, filed on Mar. 7, 2022.

(51) Int. Cl. G01N 33/574; A61K 39/00; C07K 16/28

(52) U.S. Cl. CPC G01N 33/57423; C07K 16/2827; A61K 2039/505; G01N 2333/70517; G01N 2333/70532; G01N 2333/70596

(57) ABSTRACT

Methods for treating a patient having a solid tumor are disclosed, comprising: (a) obtaining a tumor sample from the patient; (b) assessing the sample for levels of biomarkers for at least one of innate immune cells and adaptive immune cells; and (c) administering an effective amount of an immunotherapy to the patient if the sample comprises elevated levels of CD68+ PD-L1+ macrophages and CD8+ T cells compared to control and/or elevated levels of CD20+ B cells compared to control and/or low levels of CD163 expression compared to control and PD-L1 expression of greater or equal to (≥) 50%.

### FIELD OF THE INVENTION

The present disclosure generally relates to methods of treating patients having cancer.

### BACKGROUND

New anticancer therapies, such as immunologic therapies, can lead to improvements in overall survival (OS) in patients suffering from late-stage cancers, such as non-small cell lung cancer (NSCLC). However, improved outcomes and durable responses are not uniformly observed in patients treated with immunologic therapies. This inconsistency in positive results has been observed in various cancer types treated with different immunologic therapies, such as, for example, advanced NSCLC patients treated with an anti-PD-L1 immunotherapy.

Accordingly, it is of great therapeutic interest to be able to identify patients who have a greater chance of experiencing a therapeutic benefit in response to immunotherapy to allow for better treatment decisions based on a particular patient's clinical state. There is a significant need for both predictive and prognostic measures that allow for early identification of cancer patients who are likely to respond favorably to immunotherapy. Early identification of such patients is critical so that administration of immunotherapy treatments can be fast-tracked in place of the current standard of care (SOC) chemotherapy regimens. And, to the extent that such positive predictive and prognostic characteristics can be understood and possibly replicated in individuals lacking these characteristics, it may be possible to significantly improve overall survival (OS) in an even greater number of patients.

Efficacy of any given cancer treatment is heavily influenced by a tumor's intrinsic properties and the tumor microenvironment (TME). The TME is the environment created by a tumor that includes normal and cancerous cells, surrounding vasculature, infiltrating immune cells, extracellular matrix, and a multitude of signaling molecules. The TME is at least partially defined by interactions with surrounding tissues and can significantly affect the efficacy of any anti-cancer treatment.

While it is known that the TME can help determine the effectiveness of cancer treatments, there is a lack of information regarding which factors can be reliably correlated with a favorable response to immunotherapy. This is due in part to the difficulty in correlating TME status with clinically meaningful results. Thus, there is a need for new clinically relevant assessments of TME status to determine predictive and prognostic characteristics that enable reliable predictions of immunotherapy efficacy to guide treatment decisions for individual cancer patients to improve OS.

### SUMMARY

As described herein, in a first aspect, the present disclosure provides a method of treating a patient having a solid tumor. The method includes a) obtaining a tumor sample from the patient, b) assessing the sample for levels of biomarkers for at least one of innate immune cells and adaptive immune cells, and c) administering an effective amount of an immunotherapy to the patient if the sample comprises elevated levels of CD68+ PD-L1+ macrophages and CD8+ T cells compared to control and/or elevated levels of CD20+ B cells compared to control.

In one embodiment of the first aspect, the patient has non-small cell lung cancer (NSCLC). In one embodiment of the first aspect, the patient has advanced NSCLC. In one embodiment of the first aspect, the tumor sample is obtained from a biopsy or an excised tumor. In one embodiment of the first aspect, the biomarkers comprise one or more of PD-L1, PD-1, CD8, CD68, Ki67, AE1, AE3, CD20, NKp46, FOXP3, ICOS, CD66b, CD1c, and CTLA-4. In one embodiment of the first aspect, the levels of biomarkers are assessed by immunohistochemistry (IHC) and/or multiplex immunofluorescence (mIF). In one embodiment of the first aspect, the administration of the immunotherapy improves overall survival (OS) in the patient relative to a patient having a tumor without elevated levels of CD68+ PDL1+ macrophages and CD8+ T cells compared to control.

In one embodiment of the first aspect, the method further includes (d) administering a standard of care anticancer therapeutic to the patient if the sample does not comprise elevated levels of CD68+ PDL1+ macrophages and CD8+ T cells compared to control or elevated levels of CD20+ B cells compared to control; and/or (e) administering one or more chemokines, cytokines, antibodies, antigen-presenting cells, and/or synthetic scaffolds to the patient to promote the formation of intra-tumor tertiary lymphoid structures.

In one embodiment, the standard of care anticancer therapeutic comprises one or more of cisplatin, gemcitabine, methotrexate, vinblastine, doxorubicin, cisplatin (MVAC), carboplatin, a taxane, temozolomide, dacarbazine, vinflunine, docetaxel, paclitaxel, nab-paclitaxel, vemurafenib, erlotinib, afatinib, cetuximab, bevacizumab, gefitinib and pemetrexed.

In one embodiment of the first aspect or embodiments thereof, the immunotherapy comprises an immune checkpoint inhibitor. In one embodiment, the immune checkpoint inhibitor is one or more of an anti-CTLA-4 antibody, an anti-PD-1 antibody, and an anti-PD-L1 antibody. In one embodiment, the anti-CTLA-4 antibody is tremelimumab or ipilimumab. In one embodiment, the anti-PD-1 antibody is REGN2810, SHR1210, IBI308, PDR001, nivolumab, pembrolizumab, BGB-A317, BCD-100, or JS001. In one embodiment, the anti-PD-L1 antibody comprises durvalumab, avelumab, atezolizumab, KNO35 or sugemalimab. In one embodiment, the anti-PD-L1 antibody comprises durvalumab, avelumab, atezolizumab or KNO35. In one embodiment, the anti-PD-L1 antibody is durvalumab. In one embodiment, the patient is administered durvalumab at a dose of 10 mg/kg once every two weeks (Q2W). In one embodiment, the patient is administered durvalumab at a dose of 1500 mg once every four weeks (Q4W).

In one embodiment of the first aspect or embodiments thereof, at least one of cancer cell division, tumor growth, tumor size, tumor density, or tumor metastasis is reduced in the patient.

In a second aspect, the present disclosure provides a method of improving overall survival in a patient with a solid tumor. The method includes a) obtaining a tumor sample from the patient, b) assessing the sample for levels of biomarkers for at least one of innate immune cells and adaptive immune cells, and c) administering an effective amount of an immunotherapy to the patient if the sample comprises elevated levels of CD68+ PDL1+ macrophages and CD8+ T cells compared to control and/or elevated levels of CD20+ B cells compared to control.

In one embodiment of the second aspect, the patient has NSCLC. In one embodiment of the second aspect, the patient has advanced NSCLC. In one embodiment of the second aspect or embodiments thereof, the patient is administered durvalumab at a dose of 10 mg/kg Q2W. In one embodiment of the second aspect or embodiments thereof, the patient is administered durvalumab at a dose of 1500 mg Q4W. In another embodiment of the second aspect or embodiments thereof, the patient is administered durvalumab at a dose of 1500 mg Q3W.

In a third aspect, the present disclosure provides a method of treating a patient having a solid tumor, the method comprising: (a) obtaining a tumor sample from the patient; (b) assessing the sample for levels of biomarkers for at least one of innate immune cells and adaptive immune cells; and (c) administering an effective amount of an immunotherapy to the patient if the sample comprises low levels of CD163 expression compared to control and PD-L1 expression of greater or equal to (_) 50%. In one embodiment the low levels of CD163 expression are less than or equal to (≤) 30%. In another embodiment, the sample further comprises high levels of CD45 expression compared to control.

In a fourth aspect, the present disclosure provides a method of improving overall survival in a patient with a solid tumor, comprising: (a) obtaining a tumor sample from the patient; (b) assessing the sample for levels of biomarkers for at least one of innate immune cells and adaptive immune cells; and (c) administering an effective amount of an immunotherapy to the patient if the sample comprises low levels of CD163 expression compared to control, and optionally elevated levels of CD45 expression compared to control.

### BRIEF DESCRIPTION OF THE DRAWINGS

FIG. 1: Relative differences in T effector, B cell, and dendritic cell gene expression profiles between long OS and short OS groups.

FIG. 2: Relative differences in CD8, CD8 subpopulations, and CD68 subpopulations biomarker protein expression profiles between long OS and short OS groups. Elevated CD8+, CD8+ PD1+Ki67+, CD68+ PD-L1+, and CD68+ PD-L1+Ki67+ population densities are correlated with long OS.

FIG. 3: Relative differences in B cell biomarker protein expression profiles between long OS and short OS groups. Elevated B cell density indicative of TLS is correlated with long OS.

FIG. 4: Baseline Tumor of NSCLC Long OS with TLS (IHC and mIF1a).

FIG. 4 depicts a true immunological battlefield with immune cells grappling with tumor cells in a tumor of an NSCLC long OS patient. Upper left photograph: a light microscopic IHC image showing CD20 staining. High density CD20+ B cells correlates with TLS+ tumors. TLS+ enriched in 50% pts with Long OS>2 yr. Central photograph-immunofluorescent staining (with the mF1a panel) with DAPI (dark blue), CD8 (light blue), PD-L1 (green), PD-1 (yellow), CD68 (orange), and Ki67 (pink) staining. Elongated nuclear shapes in proliferation (Ki67 pink) are possibly follicular dendritic cells (FDC). Upper right photograph—an enlarged panel from the central photograph indicated via dashed lines. Arrow A-CD68+ cells (possibly antigen presenting cells (APC)) in synapsis with PD1+ cells (possibly T helpers); Arrow B-CD8+ cytotoxic T cells in synapsis with a tumoral cell; Arrow C-CD8+Ki67+, proliferating cells. Lower right photograph-CD68+ Macrophages engulfing a tumoral cell (Arrow D). Lower left photograph—an enlarged panel from the central photograph indicated via dashed lines. Arrow E—Phagocytosis near TLS, CD68+ cells with unclear/punctuated staining.

FIG. 5: High density of TLS (CD20+ B cells) predicts for OS benefit (median overall survival (mOS) not reached (NR)) compared to 16.6 months in PD-L1≥ 25% in NSCLC patients treated with durvalumab. Twenty five percent of patients (9 of 35) exhibited high CD20+ cell densities, which correlated with mOS NR.

FIG. 6: Relative measure of innate immune response in TME based on macrophage density. CD68+ PD-L1+ and CD68+ PD-L1+Ki67+ macrophage densities were significantly higher in long OS patients (n=19 for each) compared to short OS patients (N=62 and 60, respectively).

FIG. 7: Immunofluorescent image of a long OS patient tumor sample stained for the mIF1a panel. Dashed circles highlight CD68+ (orange) PD-L1+ (green) macrophages in synapsis with CD8 (light blue) T cells, while surrounding/in synapsis with tumor cells.

FIG. 8: Relative measure of adaptive immune response in TME based on CD8 T cell density. CD8+ and CD8+ PD-1+Ki67+ T cell densities were significantly higher in long OS patients (n=36 and 17, respectively) compared to short OS patients (N=129 and 59, respectively).

FIG. 9: Immune marker combination using optimized cutoff for OS. Patients with both biomarkers: high CD8+ T cells and high macrophages CD68+ PD-L1+ show significant increase in median OS (39.5 months) compared to single high biomarkers (16.6-20.2 months). These observations may provide superior predictive value for immune therapies. Curve A-high CD68+ PD-L1+ and high CD8+; Curve B-PD-L1>=25%; Curve C-PD-L1<25%; Curve D-high CD68+ PD-L1+ and low CD8+; Curve E-low CD68+ PD-L1+ and low CD8+; Curve F-low CD68+ PD-L1+ and high CD8+. PD-L1 TC status reported as % PD-L1 TC, SP263 Ab Ventana Central lab. Density PD-L1 is derived from mIF: digitally measured as cells/mm²; using SP263 Ab. Biomarker profiles are correlated with mOS and HR (95% CI).

FIG. 10: High densities of CD68+ PD-L1+ macrophages and CD8+ T cells predict for 39.5 month mOS compared to 16.6 month mOS in PD-L1≥25% NSCLC patients treated with durvalumab. Patient prevalence vs. mOS based on high marker density or combination markers—in comparison with PD-L1 TC status-based on optimized marker cutoff. CD20 is not shown as CD20+ high mOS is NR.

FIG. 11: Computational explanation for cut-off optimization-trade-off between prevalence vs. mOS/mPFS. TME: tumor microenvironment; NSCLC: non-small cell lung cancer; mOS: median overall survival; mPFS: median progression-free survival; ORR: overall response rate; HR: hazard ratio; mIF: multiplex immunofluorescence assay; Ph3: Phase 3; TLS: tertiary lymphoid structure; NR: not reached.

FIG. 12: Single biomarker: high CD68+ PD-L1+ or high CD8+ associated with OS benefit with improved mOS and HR when compared to PD-L1 TC≥25% (mOS 16.6; HR 0.55 (95% CI: 0.51-0.60)). TME: tumor microenvironment; NSCLC: non-small cell lung cancer; mOS: median overall survival; mPFS: median progression-free survival; ORR: overall response rate; HR: hazard ratio; mIF: multiplex immunofluorescence assay; Ph3: Phase 3; TLS: tertiary lymphoid structure; NR: not reached.

FIG. 13: Overall survival in NSCLC patients treated with durvalumab, analyzed by CD163, STAB1 (FIG. 13A) or C1QC protein (FIG. 13B) expression (M2 markers) and by PD-L1 status (50% cutoff). FIG. 13A: Curve A-STAB1_Low; PD-L1>50%; Curve B-STAB1_High; PD-L1>=50% Curve C-STAB1_Low; PD-L1<50% Curve D-STAB1_High; PD-L1<50%. FIG. 13B: Curve A-C1QC_Low; PD-L1>=50%; Curve B-C1QC_High; PD-L1>=50% Curve C-C1QC_Low; PD-L1<50% Curve D-C1QC_High; PD-L1<50%. High CD163 protein expression and other M2 associated markers (STAB1, C1QC) are associated with poor durvalumab benefit and even in PD-L1 TC≥50% NSCLC patients.

FIG. 14, FIG. 14A and FIG. 14B: mOS benefit derived from immune infiltration at baseline occurs only in subjects with low CD163 expression. FIG. 14A: Curve A-CD163_Low; PTPRC_High Curve B-CD163_High; PTPRC_High; Curve C-CD163_Low; PTPRC_Low; Curve D-CD163_High; PTPRC_Low. High immune infiltration (PTPRC=CD45) is associated with OS benefit. High CD45 and high CD163 is associated with poor OS benefit while high CD45 and low CD163 expression associated with OS benefit. Moreover, patients with poorer outcome have significantly higher baseline CD163 protein expression as a proportion of total CD45 protein expression (FIG. 14A).

### DETAILED DESCRIPTION

Unless defined otherwise, all technical and scientific terms used herein have the meaning commonly understood by a person skilled in the art to which this disclosure belongs. The following references provide one of skill with a general definition of many of the terms used in this disclosure: Singleton, et al., Dictionary of Microbiology and Molecular Biology (2nd ed. 1994); The Cambridge Dictionary of Science and Technology (Walker ed., 1988); The Glossary of Genetics, 5th Ed., R. Rieger, et al. (eds.), Springer Verlag (1991); and Hale & Marham, The Harper Collins Dictionary of Biology (1991). As used herein, the following terms have the meanings ascribed to them below, unless specified otherwise.

The terms “administration” and “administering” as used herein refer to providing, contacting, and/or delivering an immune therapy (or immunotherapy) or other therapeutic agent (such as a chemotherapeutic agent) by any appropriate route to achieve the desired effect in a subject. Examples of administration of a therapeutic agent can include, but are not limited to, oral, sublingual, parenteral (e.g., intravenous, subcutaneous, intracutaneous, intramuscular, intraarticular, intraarterial, intrasynovial, intrasternal, intrathecal, intralesional, or intracranial injection), transdermal, topical, buccal, rectal, vaginal, nasal, and/or ophthalmic administration. Administration of a therapeutic agent can also be via inhalation. A further form of administration of a therapeutic agent can occur via implant where a reservoir of a therapeutic agent is introduced into a subject and the therapeutic agent is released from the reservoir at a clinically relevant concentration over a predetermined period of time, such as, one or more weeks, months, or years.

The terms “co-administered” and “administered in combination” as used herein refer to simultaneous or sequential administration of multiple therapeutic compounds or agents. A first therapeutic compound or agent may be administered before, concurrently with, or after administration of a second therapeutic compound or agent. The first therapeutic compound or agent and the second therapeutic compound or agent may be simultaneously or sequentially administered on the same day, or may be sequentially administered within 1 day, 2 days, 3 days, 4 days, 5 days, 6 days, 1 week, 2 weeks, 3 weeks, or 1 month of each other. In some embodiments, therapeutic compounds or agents are co-administered during the period in which each of the therapeutic compounds or agents are exerting at least some physiological effect and/or has remaining efficacy.

As used herein, “therapeutic agent” or “therapeutic compound” can refer to a substance, such as an antibody, chemical, and/or pharmaceutical composition that when administered to a subject in need thereof in a therapeutically effective amount provides a therapeutic benefit to subject having a particular disease or disorder being treated. As used herein, “therapeutic benefit” refers to the eradication or amelioration of the underlying disease being treated and/or eradication or amelioration of one or more of the symptoms associated with the underlying disease such that a subject being treated with the therapeutic agent reports an improvement in feeling or condition, notwithstanding that the subject may still be afflicted with the underlying disease.

Therapeutic agents contemplated for use herein include immune checkpoint inhibitors, chemotherapeutic agents, and radiotherapy. Examples of immune checkpoint inhibitors include anti-PD-L1 antibodies and/or anti-CTLA4 antibodies. Other examples are described herein.

By “anti-PD-L1 antibody” is meant an antibody that selectively binds a PD-L1 polypeptide. Exemplary anti-PD-L1 antibodies are described for example at U.S. Pat. Nos. 8,779,108; 9,493,565; and 10,400,039, which are incorporated by reference for all purposes. Durvalumab (MEDI4736), or “Durva,” is an exemplary anti-PD-L1 antibody that is suitable for the methods described herein. Other anti-PD-L1 antibodies can also be used.

By “anti-CTLA4 antibody” is meant an antibody that selectively binds a CTLA4 polypeptide. Exemplary anti-CTLA4 antibodies are described for example at U.S. Pat. Nos. 6,682,736; 7,109,003; 7,132,281; 7,411,057; 7,824,679; 8,143,379; 7,807,797; and 8,491,895 (Tremelimumab is 11.2.1, therein), which are herein incorporated by reference for all purposes. Ipilimumab and Tremelimumab, or “Treme,” are exemplary anti-CTLA4 antibodies. Other anti-CTLA4 antibodies can also be used.

The terms “biomarker” and “marker” (which can be used interchangeably) as used herein generally refer to a protein, nucleic acid molecule, clinical indicator, and/or other analyte that is associated with a cell type. In one embodiment, a biomarker can be differentially expressed (or present) in a biological sample obtained from a subject having a disease (e.g., lung cancer) relative to the concentration present in a control sample or reference. In a further embodiment, a biomarker can include a measure of the gene expression of a particular gene of interest.

In another embodiment, a biomarker can be an indicator of the density of a cell type within a tissue sample. For example, when a concentration of a biomarker in a patient having a disease (e.g., cancer) is elevated compared to control (for example, a subject without cancer), then the elevated concentration can be indicative of the presence of a particular cell type, for example, an immune cell and can be indicative of the presence of an immune response associated with the disease.

In some embodiments, concentrations of one or more biomarkers or densities of cells expressing such biomarkers can be indicative of a patient's immune fitness, for example, the relative ability of the patient's immune system to combat a particular disease by itself or the relative ability of the patient's immune system to be augmented or modified to combat a particular disease, such as cancer, by treatment with an therapeutic agent such as an immune checkpoint inhibitor (ICI).

In this disclosure, the terms “comprises,” “comprising,” “containing,” “having,” and the like can have the meaning ascribed to them in U.S. Patent law and can mean “includes,” “including,” and the like; the terms “consisting essentially of” and “consists essentially of” likewise have the meaning ascribed in U.S. Patent law, and the terms are open-ended, allowing for the presence of more than that which is recited so long as basic or novel characteristics of that which is recited are not changed by the presence of more than that which is recited, but excludes prior art embodiments.

As used herein, the terms “determining,” “assessing,” “assaying,” “measuring,” “detecting,” and “identifying” refer to both quantitative and qualitative determinations, and as such, the terms can be used interchangeably. Where a quantitative determination is intended, the phrase “determining an amount” of an analyte, substance, protein, and the like can be used. Where a qualitative determination is intended, the phrase “detecting an analyte” can be used.

The term “disease” is meant any condition or disorder that damages, interferes with, or dysregulates the normal function of a cell, tissue, or organ. In a disease such as cancer (e.g., lung cancer), the normal function of a cell, tissue, or organ can be altered to enable immune evasion and/or escape of cancer cells or tumors.

The terms “immune therapy,” “immunotherapy,” and “immunologic therapy” refer to the treatment of disease by activating or suppressing the immune system. Activation immunotherapies amplify immune responses, and suppression immunotherapies reduce or suppress immune response.

By “subject” or “patient” is meant a mammal, including, but not limited to, a human, such as a human patient, a non-human primate, or a non-human mammal, such as a bovine, equine, canine, ovine, or feline animal.

As used herein, the terms “treat,” treating,” “treatment,” and the like refer to reducing, diminishing, lessening, alleviating, abrogating, or ameliorating a disorder and/or symptoms associated therewith. It will be appreciated that, although not precluded, treating a disorder or condition does not require that the disorder, condition, or symptoms associated therewith be completely eliminated.

Unless specifically stated or obvious from its context, the term “or” as used herein is understood to be inclusive. Unless specifically stated or obvious from context, the terms “a,” “an,” and “the” as used herein are understood to be singular or plural. Similarly, a particular term when expressed in the singular form also contemplates the same term expressed in plural, and vice versa. For example, the term “drug” also contemplates “drugs” and vice versa.

Unless specifically stated or obvious from context, as used herein, the term “about” is understood to refer to within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

### Overview

The present disclosure is directed to methods of treating patients with cancer, for example, advanced non-small cell lung cancer (NSCLC), to obtain greater overall survival (OS) based on an improved understanding of predictive and prognostic characteristics that are correlated with greater immunotherapy efficacy. The present disclosure provides a significant advance in cancer patient treatment methodologies because it enables clinicians to make better treatment decisions for treating cancer.

Types of cancer or “solid tumors” that are contemplated for treatment herein include, for example, NSCLC, advanced solid malignancies, biliary tract neoplasms, bladder cancer, colorectal cancer, diffuse large b-cell lymphoma, esophageal neoplasms, esophageal squamous cell carcinoma, extensive stage small cell lung cancer, gastric adenocarcinoma, gastric cancer, gastroesophageal junction cancer, head and neck cancer, head and neck squamous cell carcinoma, hepatocellular carcinoma, Hodgkin lymphoma, lung cancer, melanoma, mesothelioma, metastatic clear cell renal carcinoma, metastatic melanoma, metastatic non-cutaneous melanoma, multiple myeloma, nasopharyngeal neoplasms, non-Hodgkin lymphoma, ovarian cancer, fallopian tube cancer, peritoneal neoplasms, pleural mesothelioma, prostatic neoplasms, recurrent or metastatic PD-L1 positive or negative SCCHN, recurrent squamous cell lung cancer, renal cell cancer, renal cell carcinoma, SCCHN, hypo pharyngeal squamous cell carcinoma, laryngeal squamous cell carcinoma, small cell lung cancer, squamous cell carcinoma of the head and neck, squamous cell lung carcinoma, TNBC, transitional cell carcinoma, unresectable or metastatic melanoma, urothelial cancer, and urothelial carcinoma.

As described herein, the present disclosure features, in one embodiment, a method of treating a patient having cancer, such as non-small cell lung cancer (NSCLC) by a) obtaining a tumor sample from the patient, b) assessing the sample for levels of biomarkers for at least one of innate immune cells and adaptive immune cells, and c) administering an effective amount of an immunotherapy to the patient if the sample comprises at least one of an elevated level of an innate immune cell biomarker or an elevated level of an adaptive immune cell biomarker.

In one embodiment there is provided an immune checkpoint inhibitor for use in the treatment of a patient with a solid tumor, wherein a sample of the solid tumor comprises elevated levels of CD68+ PDL1+ macrophages and CD8+ T cells compared to control; and/or elevated levels of CD20+ B cells compared to control. In another embodiment, there is provided an anti-PDL1 antibody for use in the treatment of a patient with a solid tumor, wherein a sample of the solid tumor comprises elevated levels of CD68+ PDL1+ macrophages and CD8+ T cells compared to control; and/or elevated levels of CD20+ B cells. In another embodiment there is provided durvalumab for use in the treatment of a patient with a solid tumor, wherein a sample of the solid tumor comprises elevated levels of CD68+ PDL1+ macrophages and CD8+ T cells compared to control; and/or elevated levels of CD20+ B cells. In one embodiment the solid tumour is NSCLC. In a further embodiment, the solid tumor is advanced NSCLC.

In one embodiment there is provided an immune checkpoint inhibitor for use in the treatment of a patient with a solid tumor, wherein a sample of the solid tumor comprises low levels of CD163 expression compared to control and PD-L1 expression of greater or equal to 50%. In another embodiment, there is provided an anti-PDL1 antibody for use in the treatment of a patient with a solid tumor, wherein a sample of the solid tumor comprises low levels of CD163 expression compared to control and PD-L1 expression of greater or equal to 50%. In another embodiment there is provided durvalumab for use in the treatment of a patient with a solid tumor, wherein a sample of the solid tumor comprises low levels of CD163 expression compared to control and PD-L1 expression of greater or equal to 50%. In one embodiment low levels of CD163 expression are less than, or equal to, 30%. In another embodiment the sample further comprises high levels of CD45 expression compared to control. In one embodiment the solid tumour is NSCLC. In another embodiment, the solid tumor is advanced NSCLC.

In one embodiment there is provided a method of diagnosing a patient having a solid tumor as a candidate for receiving treatment with an immunotherapy to achieve improved overall survival. The method includes (a) obtaining a tumor sample from the patient, (b) assessing the sample for levels of biomarkers for at least one of innate immune cells and adaptive immune cells, and (c) diagnosing the patient as a candidate for receiving treatment with an immunotherapy (e.g., an immune checkpoint inhibitor) if the sample comprises elevated levels of CD68+ PD-L1+ macrophages and CD8+ T cells compared to control and/or elevated levels of CD20+ B cells compared to control. The immune checkpoint inhibitor can be any of an anti-CTLA-4 antibody, an anti-PD-1 antibody, and an anti-PD-L1 antibody, as disclosed elsewhere herein. For example, the immune checkpoint inhibitor can be durvalumab and can be administered at a dose of 10 mg/kg once every two weeks (Q2W), 1500 mg once every four weeks (Q4W) or 1500 mg once every 3 weeks (Q3W).

In some embodiments, the patient has advanced NSCLC. In some embodiments, the tumor sample can be obtained from a tumor biopsy or from a wholly or partially excised tumor. Biomarkers that can be assessed in the tumor sample can include one or more of PD-L1, PD-1, CD8, CD68, Ki67, AE1, AE3, CD20, NKp46, FOXP3, ICOS, CD66b, CD1c, and CTLA-4. Other biomarkers include CD163 (marker of suppressive (M2) macrophages) and CD45. In some embodiments, the degree or level of expression of the biomarkers can be assessed at the transcription level by measuring gene expression. In other embodiments, biomarker expression can be assessed at the protein level by immunohistochemistry (IHC) and/or multiplex immunofluorescence (mIF). In other embodiments, biomarker expression can be assessed by proteomics mass spectrometry. Other approaches for measuring biomarker protein expression are contemplated herein, as are known in the art.

In addition, biomarkers contemplated herein can include but are not limited to those indicative of T (CD3+), B (CD19+), NK (CD56+), T Naïve CD4+ and CD8+ (CD3+CD4/CD8+CD45RA+CD45RO-CCR7+), Treg/Activated (ACT; CD3+CD4+CD25hi/bright CD127low/−), TEM CD4+ (CD3+CD4+CD45RA-CD45RO+CCR7−), TCM CD4+ and CD8+ (CD3+CD4+/CD8+CD45RA-CD45RO+CCR7+), and T CD3+CD4+ICOS+ and CD4+CD38+ cells, in addition to those that are indicative of M-MDSC, granulocytes, monocytes, and neutrophils.

In a further embodiment, contemplated biomarkers can include changes in gene expression of particular genes of interest. The genes of interest can include, but are not limited to, T effector genes, natural killer (NK) cell genes, B cell genes, and dendritic cell (DC) genes. Examples include gene expression signatures for T effector associated genes (CD8A, EOMES, GZMA, GZMB, CXCL9, CXCL10, IFNG, TBX21), NK cell associated genes (NCRI (NKp48), GNLY, KLRC3, KLRD1, KLRF1, NCRI), B cell associated genes (CD19, MS4A1, CD22, CD79A), neutrophil associated genes (CD177, and DC associated genes (CDIC, KIT, CCR7, BATF3, FLT3, ZBTB46, IRF8, BTLA, and MYCL). Further biomarkers can include ARGI, IL10, HLA-DRA, and HLA-DRB1.

Measurement of gene and/or protein biomarker expression in a tumor can provide a general assessment of the relative adaptive and/or or innate immune cell presence in the tumor and provide valuable insight into how a patient may respond to immune therapy, such as, treatment with an immune checkpoint inhibitor. Indeed, while not wishing to be bound by theory, it is believed that patients having one or more tumors with at least one of an elevated level of an innate immune cell biomarker or an elevated level of an adaptive immune cell biomarker will experience greater overall survival (OS) when treated with an immunotherapy, such as durvalumab, compared to a patient without elevated levels of such biomarkers. Moreover, it believed that elevated levels of adaptive and/or innate immune cell biomarkers in tumors can be indicative of the presence of tertiary lymphoid structures (TLSs) associated with (or within the TME) of the tumors which may play a vital role for a patient experiencing improved OS.

The tertiary lymphoid structure (TLS) is an ectopic lymphoid organ that develops in non-lymphoid tissues at sites of chronic inflammation such as tumors. TLSs can mature in tumors to promote an adaptive anti-tumor immune response that translates into a clinical benefit in patients with cancer. Promotion of intra-tumor TLS formation (e.g., via treating patients with chemokines, cytokines, antibodies, antigen-presenting cells and/or synthetic scaffolds) in patients lacking intra-tumor TLSs may lead to improved treatment outcomes with immunotherapies. (Sautès-Fridman et al. Tertiary lymphoid structures in the era of cancer immunotherapy. Nat Rev Cancer 19, 307-325 (2019)).

Therefore, in instances when a patient is found to not exhibit elevated levels of adaptive and/or innate immune cell biomarkers indicative of an active antitumoral immune response and/or the presence of a TLS, then treatment decisions to administer standard of care anticancer therapeutics to the patient may be indicated. As contemplated herein, standard of care anticancer therapeutics can include one or more of cisplatin, gemcitabine, methotrexate, vinblastine, doxorubicin, cisplatin (MVAC), carboplatin, a taxane, temozolomide, dacarbazine, vinflunine, docetaxel, paclitaxel, nab-paclitaxel, vemurafenib, erlotinib, afatinib, cetuximab, bevacizumab, gefitinib, and pemetrexed. Further examples include drugs targeting DNA damage repair systems, such as poly (ADP-ribose) polymerase 1 (PARP1) inhibitors and therapeutics inhibiting WEE1 protein kinase activity, ATR protein kinase activity, ATM protein kinase activity, Aurora B protein kinase activity, and DNA-PK activity.

Still further, administration of one or more chemokines, cytokines, antibodies, antigen-presenting cells and/or synthetic scaffolds to the patient to promote the formation of intra-tumor TLSs may be indicated to improve (or indeed allow) subsequent treatment with an immunotherapy, such as durvalumab, to improve OS. Examples of contemplated TLS-promoting compounds are described, for example, in Sautès-Fridman et al. Tertiary Lymphoid Structures in Cancers: Prognostic Value, Regulation, and Manipulation for Therapeutic Intervention. Front Immunol. 7:407 (2016).

In some embodiments, contemplated immunotherapies can include an immune checkpoint inhibitor. Examples of immune checkpoint inhibitors include anti-CTLA-4 antibodies, anti-PD-1 antibodies, and anti-PD-L1 antibodies. In some embodiments, an anti-CTLA-4 antibody can be tremelimumab or ipilimumab. In some embodiments, an anti-PD-1 antibody can be REGN2810, SHR1210, IBI308, PDR001, nivolumab, pembrolizumab, BGB-A317, BCD-100, or JS001. In some embodiments, the anti-PD-L1 antibody includes durvalumab, avelumab, atezolizumab KNO35 or sugemalimab. In some embodiments, the anti-PD-L1 antibody includes durvalumab, avelumab, atezolizumab, or KNO35. In some embodiments, the anti-PD-L1 antibody is durvalumab. Any therapeutically effective antibody subparts, such as antigen-binding fragments thereof, are also contemplated herein.

In some embodiments, a treatment contemplated herein halts, reduces, slows, or otherwise lessens or improves one or more symptoms of the patient's cancer. For example, the disclosed methods can reduce the rate of cancer cell division or tumor growth, reduce tumor size or tumor density, and/or slow or halt tumor metastasis in the patient. In some embodiments, the treatment improves OS.

Further, the present disclosure features, in another embodiment, a method of improving overall survival in a patient with advanced NSCLC. The method includes a) obtaining a tumor sample from the patient, b) assessing the sample for levels of biomarkers for at least one of innate immune cells and adaptive immune cells, and c) administering an effective amount of an immunotherapy to the patient if the sample comprises elevated levels of CD68+ PDL1+ macrophages and CD8+ T cells compared to control. In one embodiment, the combination of high density CD68+ PDL1+ macrophages and CD8+ T cells is associated with improved or long OS, compared to either single biomarker.

In a further embodiment, the present disclosure features a method of improving overall survival in a patient with advanced NSCLC. The method includes a) obtaining a tumor sample from the patient, b) assessing the sample for levels of biomarkers for at least one of innate immune cells and adaptive immune cells, and c) administering an effective amount of an immunotherapy to the patient if the sample comprises elevated levels of CD20+ B cells. The elevated levels of CD20+ B cells indicate the presence of a tertiary lymphoid structure (TLS) associated with the tumor. Moreover, a high density of CD20+ B cells in the sample are indicative of TLS presence and associated with long OS.

Further, the present disclosure features, in another embodiment, a method of improving overall survival in a patient with advanced NSCLC. The method includes a) obtaining a tumor sample from the patient; (b) assessing the sample for levels of biomarkers for at least one of innate immune cells and adaptive immune cells; and

(c) administering an effective amount of an immunotherapy to the patient if the sample comprises low levels of CD163 expression compared to control, and optionally elevated levels of CD45 expression compared to control.

In some embodiments, immune therapies can be administered in one or more doses of about 1, or about 3, or about 10, or about 15 mg/kg every 1, 2, 3, or 4 weeks. For example, a patient can be treated with durvalumab at a dose of 10 mg/kg Q2W.

Further, the present disclosure features, in another embodiment, use of an immunotherapy for the manufacture of a medicament for treating a patient having a solid tumor, the use comprising: (a) obtaining a tumor sample from the patient; (b) assessing the sample for levels of biomarkers for at least one of innate immune cells and adaptive immune cells; and (c) administering an effective amount of the immunotherapy to the patient if the sample comprises elevated levels of CD68+ PD-L1+ macrophages and CD8+ T cells compared to control and/or elevated levels of CD20+ B cells compared to control.

In another embodiment, the disclosure provides, use of an immunotherapy for the manufacture of a medicament for treating a patient having a solid tumor, the use comprising: (a) obtaining a tumor sample from the patient; (b) assessing the sample for levels of biomarkers for at least one of innate immune cells and adaptive immune cells; and (c) administering an effective amount of the immunotherapy to the patient if the sample comprises low levels of CD163 expression compared to control and PD-L1 expression of greater or equal to (≥) 50%.

The amount of anticancer therapeutics, such as antibodies or antigen-binding fragments thereof to be administered to a patient will depend on various parameters such as the patient's age, weight, clinical assessment, immune fitness, TME status, tumor burden and/or other factors, including the judgment of the attending physician. Any acceptable route of administration is contemplated, such as, without limitation, administration intravenous (e.g., intravenous infusion), parenteral, or subcutaneous routes of administration.

Any therapeutic compositions or methods contemplated herein can be combined with one or more of any of the other therapeutic compositions and methods provided herein.

In some embodiments, a treatment regimen can include a biological component, such as an antibody and one or more of a TLS promoting component and a chemotherapeutic component.

In some embodiments, a kit is contemplated including a biological component, such as an antibody and one or more of a TLS promoting component and a chemotherapeutic component.

### Examples

The Examples that follow are illustrative of specific embodiments of the invention, and various uses thereof. They are set forth for explanatory purposes only and should not be taken as limiting the scope of the disclosure in any way.

### Example 1-Presence of Tertiary Lymphoid Structure (TLS) and Combined High Densities of PD-L1+ Macrophages and CD8+ T Cells Predicts Long-Term Overall Survival (OS) for Durvalumab-Treated Patients with Advanced Non-Small Cell Lung Cancer (NSCLC)

### SUMMARY

Baseline PD-L1 expression has demonstrated its clinical utility in predicting OS in NSCLC patients receiving anti-PD-(L) 1 therapies including durvalumab. However, beyond the Tumor PD-L1-T cell axis, and in light of the interplay between innate and adaptive cells, the impact of the tumor microenvironment (TME) and immune contexture (e.g., relative numbers of immune cell types present) deserves to be investigated in the quest for better predictor(s) of durvalumab overall survival for patient selection. To do so, we sought to determine the biomarkers of the TME/immune contexture associated with long OS>2 yr vs. short OS<1 yr in NSCLC patients treated with durvalumab using RNA sequencing (RNAseq). As a result of the RNAseq analysis we developed multiplex immunofluorescence (mIF1a/b) and IHC to explore the overall immune contexture including tertiary lymphoid structure (TLS).

Greater pre-existing adaptive (T, B cells) and innate (MACs, DCs and NK) immunity, including TLS show strong association with durvalumab treated NSCLC patients with long OS (>2 yr) vs. short OS (<1 yr). Fifty percent of NSCLC Long OS patients with high density of CD20+ cells show the presence of TLS with increased APCs-T-cells and immune cell-tumor synapsis. Nearby the TLS an active anti-tumor immune response via phagocytosis is observed. These findings highlight the important interplay between innate-adaptive immune cells and Immune-Tumor cells and suggest its relationship to TLS presence.

### INTRODUCTION

Predictive biomarkers of anti-PD-(L) 1 therapies (e.g., durvalumab) have largely focused on the tumor-T cell axis where tumor cell PD-L1 expression has demonstrated its clinical utility in predicting overall survival (OS) in patients with advanced non-small cell lung cancer (NSCLC). Although, other immune cell subsets were shown to be associated with clinical efficacy, their relative impact and combined effect in predicting improved long-term survival warrant further investigation. Using computational image analysis of multiplex immunofluorescence (mIF) and immunohistochemistry (IHC) immune marker panels, we sought to identify single and combined biomarkers of the tumor immune contexture in association with long-term OS in advanced NSCLC patients treated with Durvalumab.

### Methods

Pre-treatment tumor samples from advanced NSCLC patients (n=210) enrolled in durvalumab nonrandomized phase ½ trial (10 mg/kg Q2W, CP1108/NCT01693562) were analyzed via RNAseq to identify candidate biomarkers of the TME/immune contexture associated with long OS vs. short OS.

Next, tumor samples were stained using IHC and 6-marker mIF panels developed as a result of the RNAseq analysis to detect markers of immune cells, cell functional state, and tertiary lymphoid structure (TLS). The panels are shown in Table No. 1. Cell marker density (cells/mm²), distribution, and proximity were quantified and analyzed in association with OS.

TABLE NO. 1
Biomarker panels for IHC and mIF.

| mIFa (immune | mIFb (immune | Single/ |
|---|---|---|
| proliferation) | contexture) | duplex IHC |
| PD-L1 | CD20 | PD-L1 |
| PD-1 | NKp46 | CD8 |
| CD8 | FOXP3 | CD20/Nkp46 |
| CD68 | ICOS | CTLA-4/FOXP3 |
| Ki67 | CD66b | |
| AE1/AE3 | CD1c | |

### Results

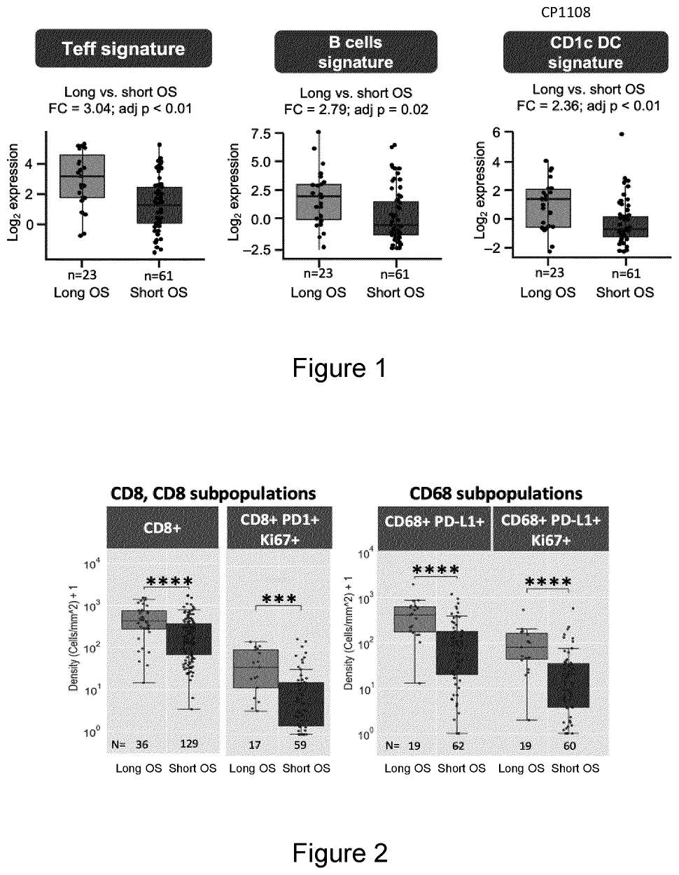

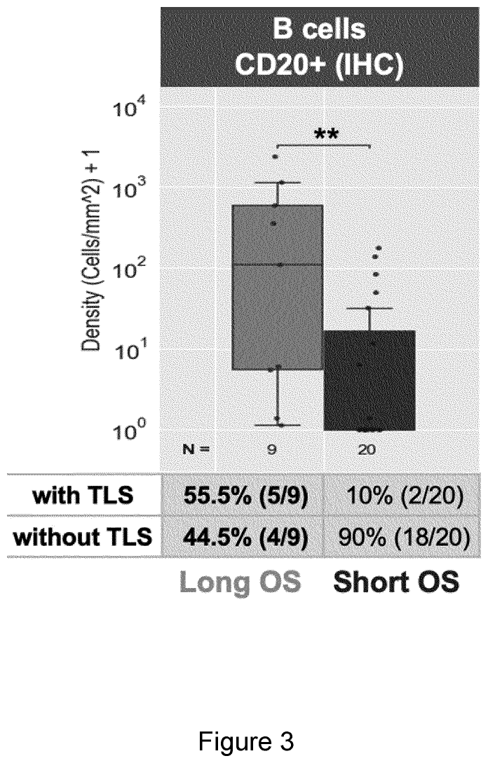

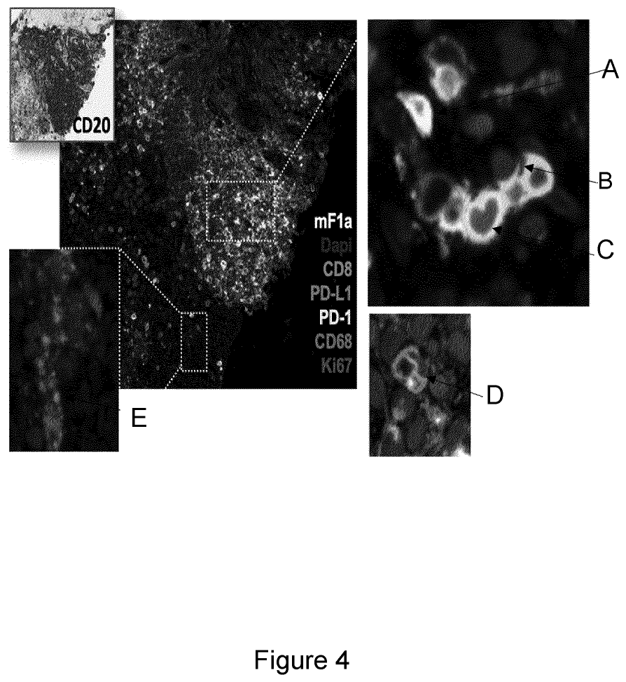

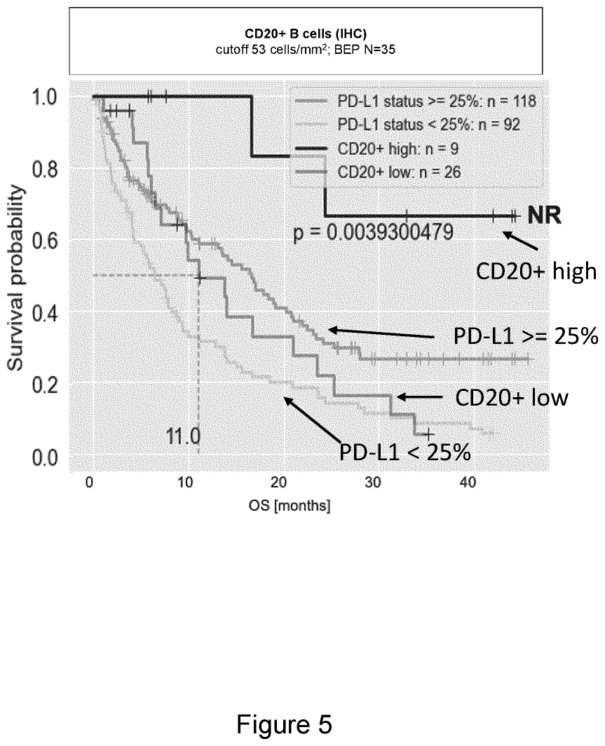

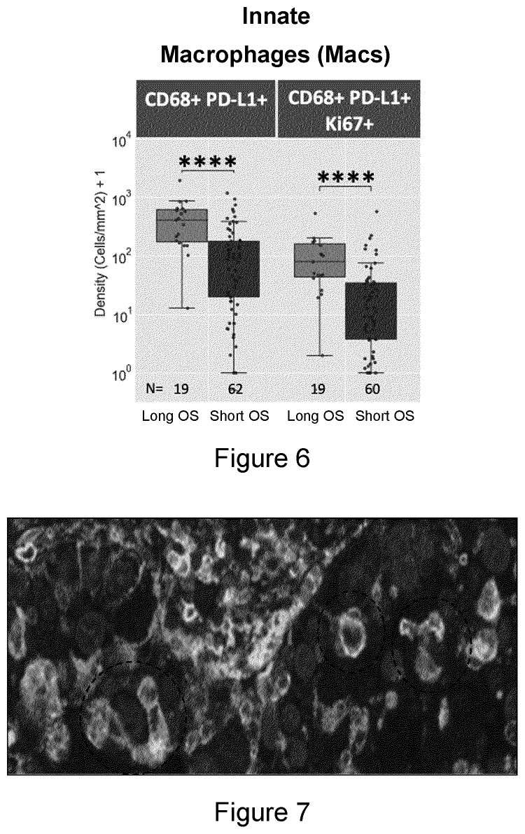

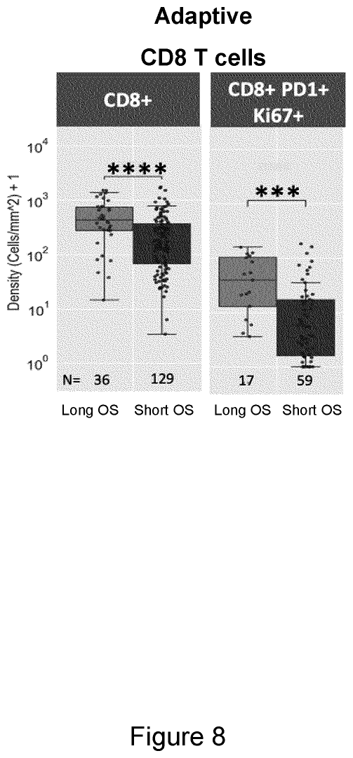

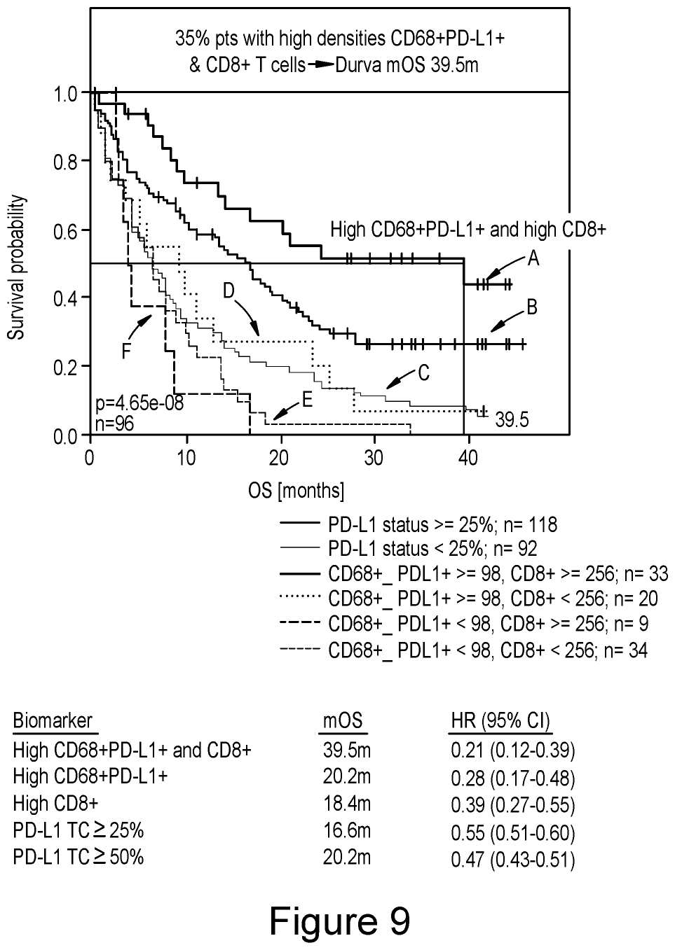

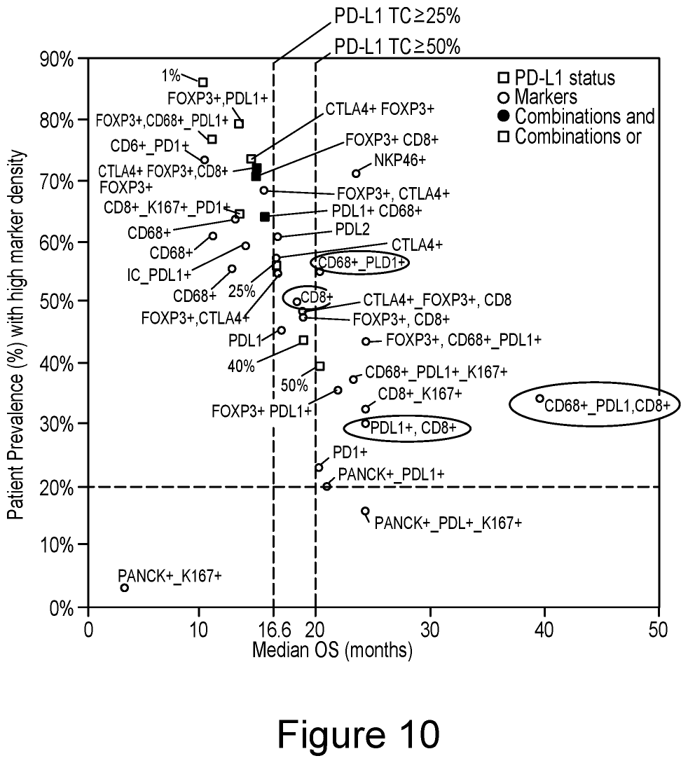

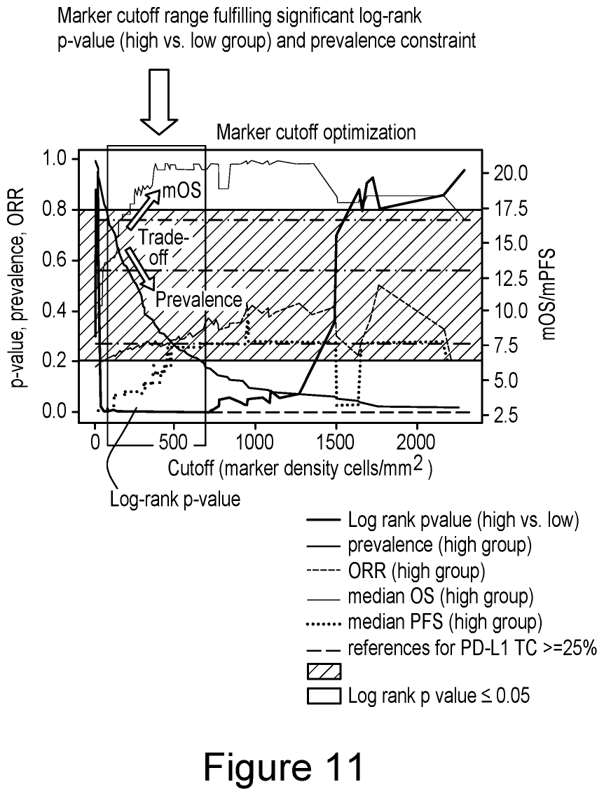

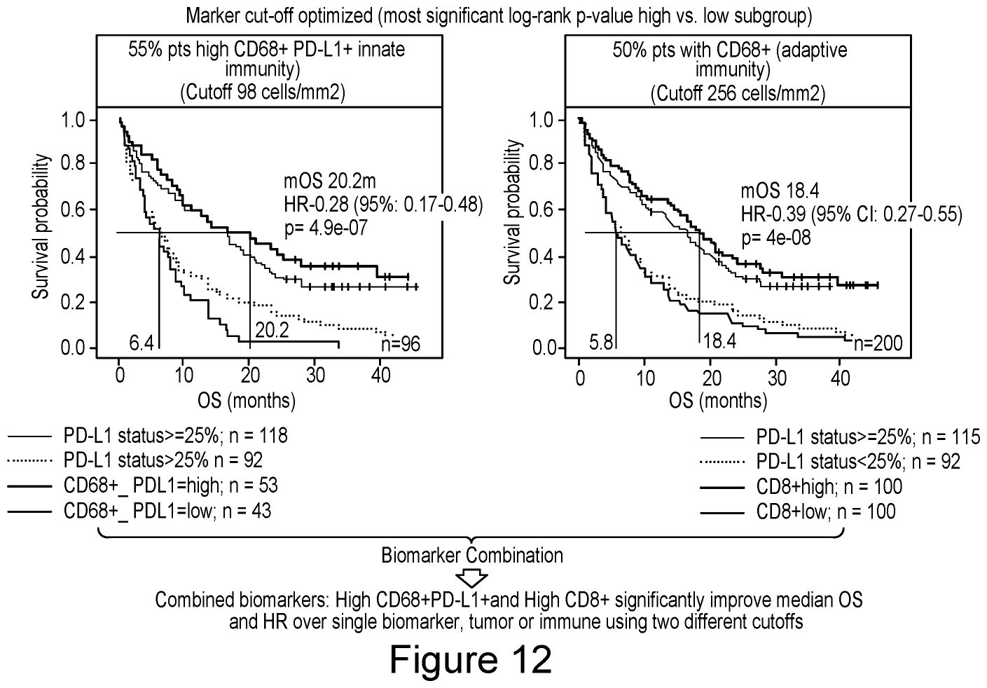

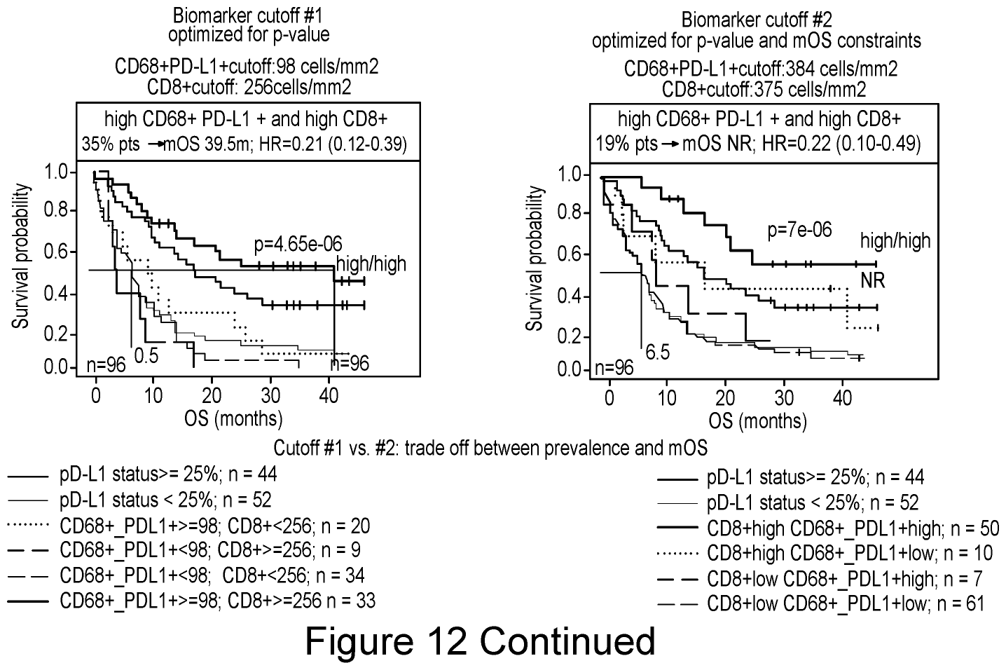

Significant differences in T effector, B cell, and dendritic cell gene expression profiles were observed between long OS and short OS groups (FIG. 1). As shown in FIGS. 2-12, computational image analysis of the tumor immune contexture revealed a greater immune inflamed phenotype, both innate (macrophages, dendritic cells) and adaptive (T and B cells), in NSCLC patients with long OS compared to those with short OS (fold change>2, p<0.0001). Patient subgroup with high density of individual immune subsets show a median OS (mOS) of 10-20 months (p<0.01 high vs. low subgroups) while combined markers of innate and adaptive immune cells show an improved mOS>2 years (p<0.001).

Specifically among the key findings, combined biomarkers of high density of CD68+ PD-L1+ macrophages and high density of CD8+ T cells predict for a significant increase in mOS of 39.5 months (p-value<10-7, HR=0.21, 95% CI 0.12-0.39) compared to mOS of either single biomarker, CD68+ PD-L1+ macrophage (20.2 month mOS, p<106, HR=0.28, 95% CI 0.17-0.48) or CD8+ T cells (18.4 month mOS, p<10-7, HR=0.39, 95% CI 0.27-0.55). Moreover, a high density of CD20+ cells, reflective of B cell tumor infiltration and the presence of a TLS, predicts for long-term OS (mOS NR, p=0.003). TLS enriched tumors show an increased level of macrophages expressing PD-L1 in synapsis with CD8+ T cells (activated PD1+ or proliferative Ki67+ T cells).

### DISCUSSION

The present findings demonstrate the importance of both tertiary lymphoid structure and high pre-existing innate-adaptive immunity in driving long-term overall survival of durvalumab-treated patients with NSCLC and highlight the need for the development of multiparametric predictive biomarkers beyond the tumor-T cell axis.

### Example 2-Functional Status of Tumor-Associated Macrophages Impacts Clinical Outcome of Durvalumab in Patients with NSCLC as Revealed by Proteomics Mass Spectrometry

### INTRODUCTION

In Example 1, we used computational image analysis of multiplex immunofluorescence (mIF), to show the positive impact of CD68+ PD-L1+ macrophages in combination with CD8+ T cells in predicting long-term OS benefit in NSCLC patients treated with durvalumab (anti-PD-L1), highlighting the impact of the myeloid compartment on IO responses. In part, using proteomics mass spectrometry we sought to investigate further the functional impact of myeloid cells on the IO response and in particular, suppressive (M2) tumor associated macrophages (TAMs).

### Methods

Pre-treatment tumor samples from 66 patients with advanced NSCLC patients enrolled in durvalumab non-randomized phase ½ trial (10 mg/kg Q2W, CP1108/NCT01693562) were processed for global and targeted proteomics. A pathologist determined the tumor area and a single FFPE tumor tissue section was used for laser-capture macro dissection and protein extraction followed by mass spectrometry analysis using label-free, data-independent and parallel reaction monitoring.

### Results

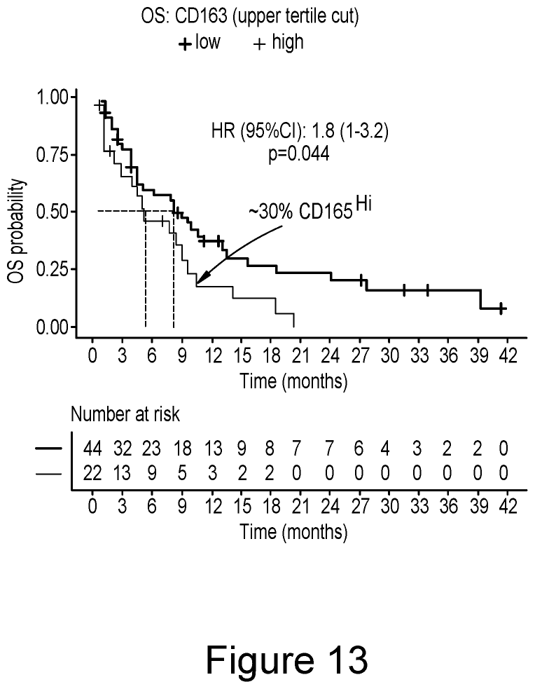

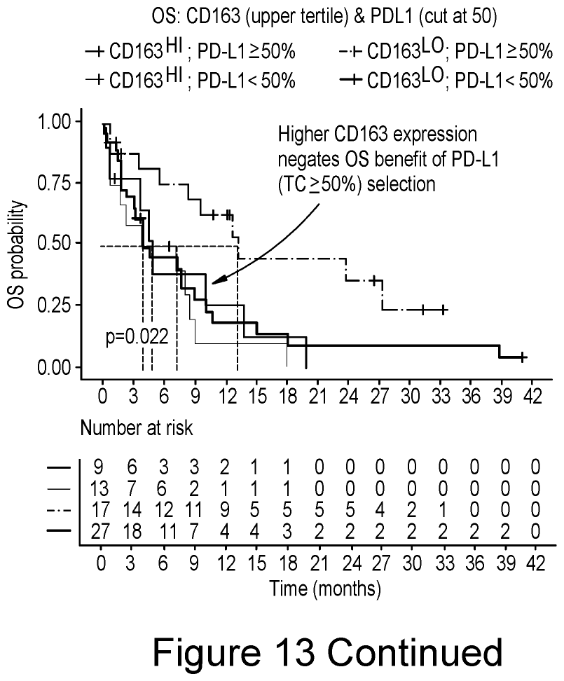

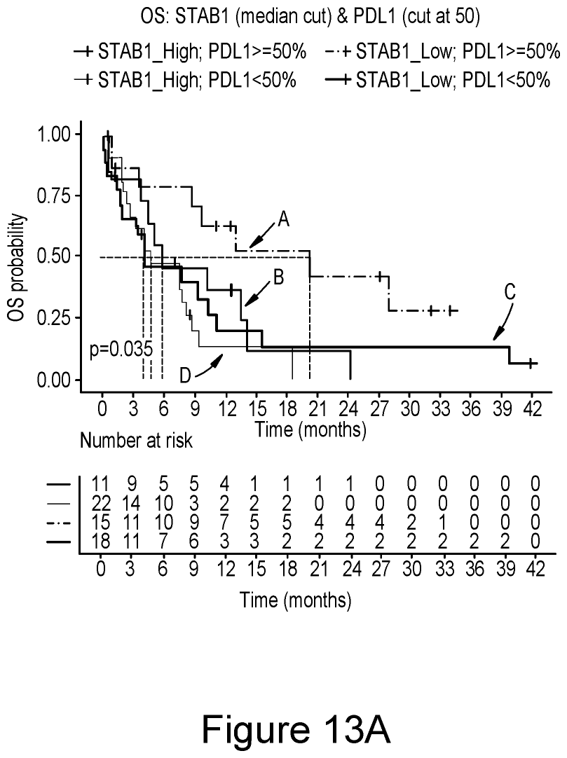

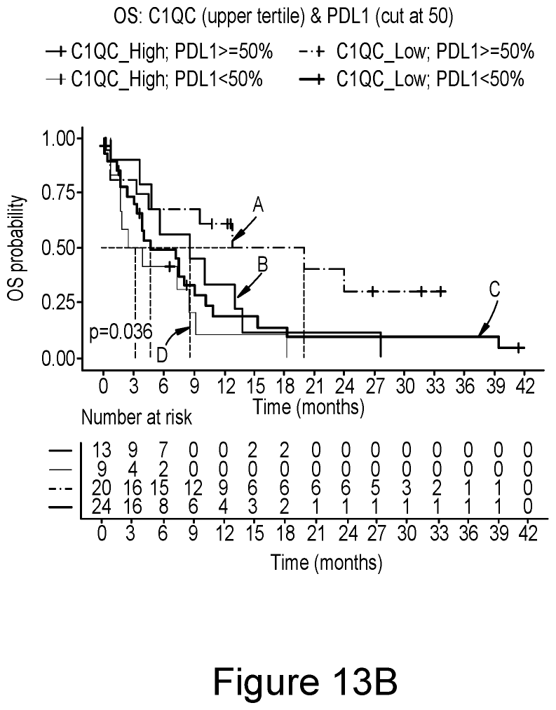

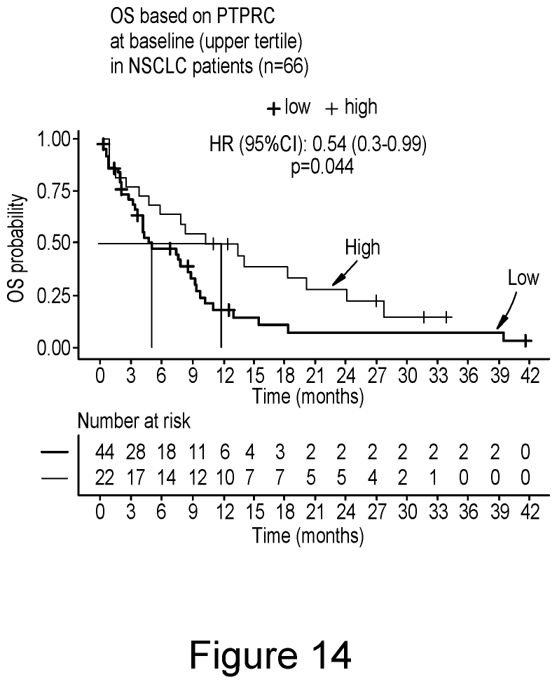

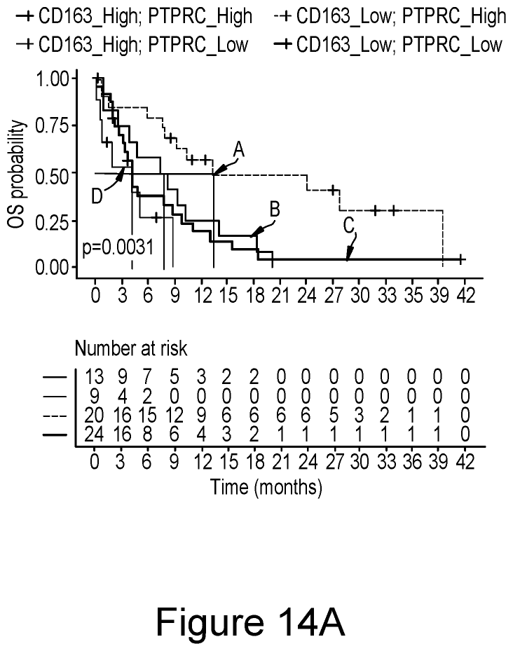

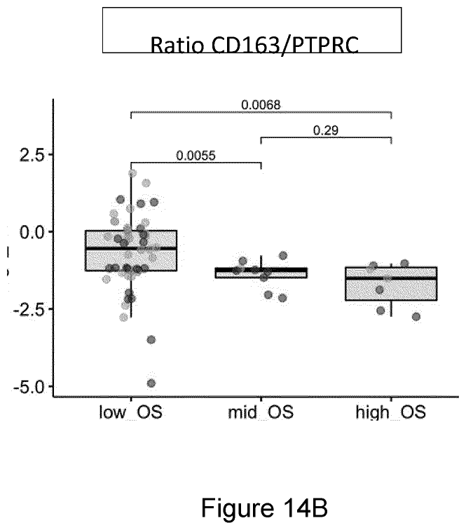

Among the immune associated proteins detected by proteomics mass spectrometry, we evaluated CD163 protein expression, a well-described marker of suppressive (M2) macrophages, and its impact on durvalumab clinical outcomes, alone and in relationship with PD-L1 and immune infiltration. While proteomics-based biomarker evaluable population (BEP) shows shorter median (m) OS compared to intended to treat population, we validated that high expression of PD-L1 as measured by IHC or proteomics associates with greater OS benefit as expected when treating with durvalumab. Building on this, the evaluation of protein associated with suppressive M2 macrophages, revealed that approximately 30% of NSCLC patients with high CD163 protein expression show poor OS benefit (5 months mOS) following durvalumab treatment. High CD163 protein expression and other M2 associated markers (STAB1, C1QC) are associated with poor durvalumab benefit and even in PD-L1 TC≥50% NSCLC patients (see FIG. 13). In contrast, patients with PD-L1 TC≥50% and low CD163 expression derive greater OS benefit (13.4 months mOS) than those with high CD163 expression (5 months mOS) or with PD-L1 TC<50% regardless of level of CD163 expression (4.1-7.4 months mOS). Moreover, patients with poorer outcome have significantly higher baseline CD163 protein expression as a proportion of total CD45 protein expression.

### CONCLUSION

This analysis further confirms the importance of myeloid cell function within the tumor microenvironment (TME) in determining the outcome to T cell-directed Immuno-oncology (IO) therapy and highlights the utility of proteomics mass spectrometry in assessing more broadly and quantitatively the TME complementing findings based on RNAseq and IHC/mIF assays.

All patents and publications mentioned in this specification are herein incorporated by reference to the same extent as if each independent patent and publication was specifically and individually indicated to be incorporated by reference. Citation or identification of any reference in any section of this application shall not be construed as an admission that such reference is available as prior art to the present disclosure.

What is claimed is:

1. An immunotherapy for use in a method of treating a patient having a solid tumor, the method comprising:
(a) obtaining a tumor sample from the patient;
(b) assessing the sample for levels of biomarkers for at least one of innate immune cells and adaptive immune cells; and
(c) administering an effective amount of the immunotherapy to the patient if the sample comprises elevated levels of CD68+ PD-L1+ macrophages and CD8+ T cells compared to control and/or elevated levels of CD20+ B cells compared to control.

2. The immunotherapy for use of claim 1, wherein the patient has non-small cell lung cancer (NSCLC).

3. The immunotherapy for use of claim 2, wherein the patient has advanced NSCLC.

4. The immunotherapy for use of any one of the preceding claims, wherein the tumor sample is obtained from a biopsy or an excised tumor.

5. The immunotherapy for use of any one of the preceding claims, wherein the levels of biomarkers are assessed by immunohistochemistry (IHC) and/or multiplex immunofluorescence (mIF).

6. The immunotherapy for use of any one of the preceding claims, wherein the administration of the immunotherapy improves overall survival (OS) in the patient relative to a patient having a tumor without elevated levels of CD68+ PDL1+ macrophages and CD8+ T cells compared to control.

7. The immunotherapy for use of any one of the preceding claims, further comprising:
(d) administering a standard of care anticancer therapeutic to the patient if the sample does not comprise elevated levels of CD68+ PDL1+ macrophages and CD8+ T cells compared to control or elevated levels of CD20+ B cells compared to control; and/or
(e) administering one or more chemokines, cytokines, antibodies, antigen-presenting cells, and/or synthetic scaffolds to the patient to promote the formation of intra-tumor tertiary lymphoid structures.

8. An immunotherapy for use in a method of treating a patient having a solid tumor, the method comprising:
(a) obtaining a tumor sample from the patient;
(b) assessing the sample for levels of biomarkers for at least one of innate immune cells and adaptive immune cells; and
(c) administering an effective amount of the immunotherapy to the patient if the sample comprises low levels of CD163 expression compared to control and PD-L1 expression of greater or equal to (≥) 50%.

9. The immunotherapy for use of claim 8, wherein low levels of CD163 expression are less than or equal to (≤) 30%.

10. The immunotherapy for use of claim 8 or claim 9, wherein the sample further comprises high levels of CD45 expression compared to control.

11. The immunotherapy for use of any one of claims 8 to 10, wherein the patient has non-small cell lung cancer (NSCLC).

12. The immunotherapy for use of claim 11, wherein the patient has advanced NSCLC.

13. The immunotherapy for use of any one of claims 8 to 12, wherein the tumor sample is obtained from a biopsy or an excised tumor.

14. The immunotherapy for use of any one of claims 8 to 13, wherein the levels of biomarkers are assessed by immunohistochemistry (IHC) and/or multiplex immunofluorescence (mIF) and/or proteomics mass spectrometry.

15. The immunotherapy for use of any one of claims 8 to 14, wherein the administration of the immunotherapy improves overall survival (OS) in the patient relative to a patient having a tumor with high levels of CD163 protein expression compared to control.

16. The immunotherapy for use of claim 15, wherein the high level of CD163 protein expression is greater than 30%.

17. The immunotherapy for use of any one of claims 8 to 16, further comprising:
(d) administering a standard of care anticancer therapeutic to the patient if the sample comprises high levels of CD163 expression compared to control; and/or
(e) administering one or more chemokines, cytokines, antibodies, antigen-presenting cells, and/or synthetic scaffolds to the patient to promote the formation of intra-tumor tertiary lymphoid structures.

18. The immunotherapy for use of claim 7 or claim 17, wherein the standard of care anticancer therapeutic comprises one or more of cisplatin, gemcitabine, methotrexate, vinblastine, doxorubicin, cisplatin (MVAC), carboplatin, a taxane, temozolomide, dacarbazine, vinflunine, docetaxel, paclitaxel, nab-paclitaxel, vemurafenib, erlotinib, afatinib, cetuximab, bevacizumab, gefitinib, and pemetrexed.

19. The immunotherapy for use of any one of the preceding claims, wherein the immunotherapy comprises an immune checkpoint inhibitor.

20. The immunotherapy for use of claim 19, wherein the immune checkpoint inhibitor is one or more of an anti-CTLA-4 antibody, an anti-PD-1 antibody and an anti-PD-L1 antibody.

21. The immunotherapy for use of claim 20, wherein the anti-CTLA-4 antibody is tremelimumab or ipilimumab.

22. The immunotherapy for use of claim 20, wherein the anti-PD-1 antibody is REGN2810, SHR1210, IBI308, PDR001, nivolumab, pembrolizumab, BGB-A317, BCD-100, or JS001.

23. The immunotherapy for use of 20, wherein the anti-PD-L1 antibody is durvalumab, avelumab, atezolizumab, KNO35 or sugemalimab.

24. The immunotherapy for use of 23, wherein the anti-PD-L1 antibody is durvalumab.

25. The immunotherapy for use of claim 24, wherein the patient is administered durvalumab at a dose of 10 mg/kg once every two weeks (Q2W).

26. The immunotherapy for use of claim 24, wherein the patient is administered durvalumab at a dose of 1500 mg once every four weeks (Q4W).

27. The immunotherapy for use of claim 24, wherein the patient is administered durvalumab at a dose of 1500 mg once every three weeks (Q3W).

28. The immunotherapy for use of any one of the preceding claims, wherein at least one of cancer cell division, tumor growth, tumor size, tumor density, or tumor metastasis is reduced in the patient.

29. A method of treating a patient having a solid tumor, comprising:
(a) obtaining a tumor sample from the patient;
(b) assessing the sample for levels of biomarkers for at least one of innate immune cells and adaptive immune cells; and
(c) administering an effective amount of an immunotherapy to the patient if the sample comprises elevated levels of CD68+ PD-L1+ macrophages and CD8+ T cells compared to control and/or elevated levels of CD20+ B cells compared to control.

30. The method of claim 29, wherein the patient has non-small cell lung cancer (NSCLC).

31. The method of claim 30, wherein the patient has advanced NSCLC.

32. The method of any one of claims 29 to 31, wherein the tumor sample is obtained from a biopsy or an excised tumor.

33. The method of any one of claims 29 to 32, wherein the levels of biomarkers are assessed by immunohistochemistry (IHC) and/or multiplex immunofluorescence (mIF).

34. The method of any one of claims 29 to 33, wherein the administration of the immunotherapy improves overall survival (OS) in the patient relative to a patient having a tumor without elevated levels of CD68+ PDL1+ macrophages and CD8+ T cells compared to control.

35. The method of any one of claims 29 to 34, further comprising:
(d) administering a standard of care anticancer therapeutic to the patient if the sample does not comprise elevated levels of CD68+ PDL1+ macrophages and CD8+ T cells compared to control or elevated levels of CD20+ B cells compared to control; and/or
(e) administering one or more chemokines, cytokines, antibodies, antigen-presenting cells, and/or synthetic scaffolds to the patient to promote the formation of intra-tumor tertiary lymphoid structures.

36. A method of treating a patient having a solid tumor, the method comprising:
(a) obtaining a tumor sample from the patient;
(b) assessing the sample for levels of biomarkers for at least one of innate immune cells and adaptive immune cells; and
(c) administering an effective amount of an immunotherapy to the patient if the sample comprises low levels of CD163 expression compared to control and PD-L1 expression of greater or equal to (≥) 50%.

37. The method of claim 36, wherein low levels of CD163 expression are less than or equal to (≤) 30%.

38. The method of claim 36 or claim 37, wherein the sample further comprises high levels of CD45 expression compared to control.

39. The method of any one of claims 36 to 38, wherein the patient has non-small cell lung cancer (NSCLC).

40. The method of claim 39, wherein the patient has advanced NSCLC.

41. The method of any one of claims 36 to 40, wherein the tumor sample is obtained from a biopsy or an excised tumor.

42. The method of any one of claims 36 to 41, wherein the levels of biomarkers are assessed by immunohistochemistry (IHC) and/or multiplex immunofluorescence (mIF) and/or proteomics mass spectrometry.

43. The method of any one of claims 36 to 42, wherein the administration of the immunotherapy improves overall survival (OS) in the patient relative to a patient having a tumor with high levels of CD163 expression compared to control.

44. The method of claim 43, wherein the high level of CD163 expression is greater than 30%.

45. The method of any one of claims 36 to 44, further comprising:
(d) administering a standard of care anticancer therapeutic to the patient if the sample comprises high levels of CD163 expression compared to control; and/or
(e) administering one or more chemokines, cytokines, antibodies, antigen-presenting cells, and/or synthetic scaffolds to the patient to promote the formation of intra-tumor tertiary lymphoid structures.

46. The method of claim 35 or claim 45, wherein the standard of care anticancer therapeutic comprises one or more of cisplatin, gemcitabine, methotrexate, vinblastine, doxorubicin, cisplatin (MVAC), carboplatin, a taxane, temozolomide, dacarbazine, vinflunine, docetaxel, paclitaxel, nab-paclitaxel, vemurafenib, erlotinib, afatinib, cetuximab, bevacizumab, gefitinib, and pemetrexed.

47. The method of any one of claims 29 to 46, wherein the immunotherapy comprises an immune checkpoint inhibitor.

48. The method of claim 47, wherein the immune checkpoint inhibitor is one or more of an anti-CTLA-4 antibody, an anti-PD-1 antibody, and an anti-PD-L1 antibody.

49. The method of claim 48, wherein the anti-CTLA-4 antibody is tremelimumab or ipilimumab.

50. The method of claim 48, wherein the anti-PD-1 antibody is REGN2810, SHR1210, IBI308, PDR001, nivolumab, pembrolizumab, BGB-A317, BCD-100, or JS001.

51. The method of claim 48, wherein the anti-PD-L1 antibody comprises durvalumab, avelumab, atezolizumab, KNO35 or sugemalimab.

52. The method of claim 51, wherein the anti-PD-L1 antibody is durvalumab.

53. The method of claim 52, wherein the patient is administered durvalumab at a dose of 10 mg/kg once every two weeks (Q2W).

54. The method of claim 52, wherein the patient is administered durvalumab at a dose of 1500 mg once every four weeks (Q4W).

55. The method of claim 52, wherein the patient is administered durvalumab at a dose of 1500 mg once every three weeks (Q3W).

56. The method of any of claims 29 to 55, wherein at least one of cancer cell division, tumor growth, tumor size, tumor density, or tumor metastasis is reduced in the patient.

57. A method of improving overall survival in a patient with a solid tumor, comprising:
(a) obtaining a tumor sample from the patient;
(b) assessing the sample for levels of biomarkers for at least one of innate immune cells and adaptive immune cells; and
(c) administering an effective amount of an immunotherapy to the patient if the sample comprises elevated levels of CD68+ PDL1+ macrophages and CD8+ T cells compared to control, and/or elevated levels of CD20+ B cells compared to control.

58. A method of improving overall survival in a patient with a solid tumor, comprising:
(a) obtaining a tumor sample from the patient;
(b) assessing the sample for levels of biomarkers for at least one of innate immune cells and adaptive immune cells; and
(c) administering an effective amount of an immunotherapy to the patient if the sample comprises low levels of CD163 expression compared to control, and optionally elevated levels of CD45 expression compared to control.

59. The method of claim 57 or claim 58, wherein the patient has NSCLC.

60. The method of claim 59, wherein the patient has advanced NSCLC.

61. The method of any one of claims 57 to 60, wherein the patient is administered durvalumab at a dose of 10 mg/kg Q2W.

62. The method of any one of claims 57 to 60, wherein the patient is administered durvalumab at a fixed dose of 1500 mg Q4W.

63. The method of any one of claims 57 to 60, wherein the patient is administered durvalumab at a fixed dose of 1500 mg Q3W.

64. Use of an immunotherapy for the manufacture of a medicament for treating a patient having a solid tumor, the use comprising:
(a) obtaining a tumor sample from the patient;
(b) assessing the sample for levels of biomarkers for at least one of innate immune cells and adaptive immune cells; and
(c) administering an effective amount of the immunotherapy to the patient if the sample comprises elevated levels of CD68+ PD-L1+ macrophages and CD8+ T cells compared to control and/or elevated levels of CD20+ B cells compared to control.

65. Use of an immunotherapy for the manufacture of a medicament for treating a patient having a solid tumor, the use comprising:
(a) obtaining a tumor sample from the patient;
(b) assessing the sample for levels of biomarkers for at least one of innate immune cells and adaptive immune cells; and
(c) administering an effective amount of the immunotherapy to the patient if the sample comprises low levels of CD163 expression compared to control and PD-L1 expression of greater or equal to (≥) 50%.
